(19) United States
(12) Patent Application Publication
(10) Pub. No.: US 2006/0135857 A1
(43) Pub. Date: Jun. 22, 2006

(54) Input device

(76) Inventors: Sheng-Chin Ho, Taipei (TW); Yu-Lin Lin, Taipei (TW)

Correspondence Address: Dougherty & Troxell;ONE SKYLINE PLACE, SUITE 1404, 5205 LEESBURG PIKE, FALLS CHURCH, VA, 22041, US

(21) Appl. No.: 11/047,572

(22) Filed: Feb. 2, 2005

(30) Foreign Application Priority Data
Dec. 20, 2004 (TW) 093220493
Dec. 24, 2004 (CN) 200420116671.6

(51) Int. Cl. A61B 5/00; A61B 5/02; A61B 5/05

(52) U.S. Cl. 600/300; 600/549; 600/485; 600/500; 600/547

(57) ABSTRACT

An input device comprises a body, having an upper cover as well as a lower cover and a space formed between them for disposing an electronic circuit with a connecting interface; a meter, disposing in the body; and a control circuit, disposing in the space and coupling to the meter and the electronic circuit; whereby a human physiology parameter detected by the meter can be transmitted to a monitor program installed on a computer through the connecting interface. Wherein, the meter can be a temperature meter, a contact meter or an infrared meter, a blood pressure and heartbeat meter, a body fat meter or a skin analyzer, such that the input device of the present invention has the ability of measuring the temperature, blood pressure and heartbeat, body fat or skin quality of human being.

### BACKGROUND OF THE INVENTION

1. Field of the Invention

The present invention relates to an input device, and particularly the present invention relates to an input device which comprises a temperature meter, a blood pressure and heartbeat meter, a body fat meter or a skin analyzer, such that the input device of the present invention has the ability of measuring the temperature, blood pressure and heartbeat, body fat or skin quality of the user when he/she uses the input device.

2. Description of Related Art

The input device, such as mouse or keyboard, is generally worked as the computer-inputted (executed) or menu-selected tool. Accompanying the computer becoming more popularity, the user uses computer more frequently during his/her office hours. However, mouse or keyboard is a maturely product with a very low price, therefore the prior art mouse or keyboard only has input function but doesn't provide any function that user can measure his/her temperature, blood pressure and heartbeat, body fat or skin quality etc. physiology parameters.

There is therefore a need for a new and improved strategy and technique for overcoming these deficiencies and the present invention overcomes these deficiencies in a new and novel fashion.

### SUMMARY OF THE INVENTION

The present invention generally relates to a strategy and a technique which overcomes some or all of the above referenced deficiencies associated with prior portable input device.

It is a non-limited object of the present invention is to provide an input device which comprises a temperature meter, a blood pressure and heartbeat meter, a body fat meter or a skin analyzer, such that the input device of the present invention has the ability of measuring the temperature, blood pressure and heartbeat, body fat or skin quality of the user when he/she uses the input device:

According to a non-limited object of the present invention, an input device is provided and comprises a body, having an upper cover as well as a lower cover and a space formed between them for disposing an electronic circuit with a connecting interface; a meter, disposing in the body; and a control circuit, disposing in the space and coupling to the meter and the electronic circuit; whereby a human physiology parameter detected by the meter can be transmitted to a monitor program installed on a computer through the connecting interface.

### BRIEF DESCRIPTION OF THE DRAWINGS

The present invention can be more fully understood by referring to the following description and accompanying drawings, in which:

FIG. 1 is the prospective view which shows a disassembly view of an input device according to one preferred embodiment of the present invention;

FIG. 2 is the prospective view which shows an assembly view of a mouse with temperature sensing function according to one preferred embodiment of the present invention;

FIG. 3 is the prospective view which shows an assembly view of a mouse with blood pressure and heartbeat sensing function according to another preferred embodiment of the present invention;

FIG. 4 is the prospective view which shows an assembly view of a mouse with body fat sensing function according to still one another preferred embodiment of the present invention;

FIG. 5 is the prospective view which shows an assembly view of a mouse with skin quality sensing function according to still one another preferred embodiment of the present invention;

FIG. 6 is the prospective view which shows a disassembly view of a keyboard with temperature sensing function according to one preferred embodiment of the present invention;

FIG. 7 is the prospective view which shows an assembly view of a keyboard with temperature sensing function according to one preferred embodiment of the present invention;

FIG. 8 is the prospective view which shows an assembly view of a keyboard with blood pressure and heartbeat sensing function according to another preferred embodiment of the present invention;

FIG. 9 is the prospective view which shows an assembly view of a keyboard with body fat sensing function according to still one another preferred embodiment of the present invention; and

FIG. 10 is the prospective view which shows an assembly view of a keyboard with skin quality sensing function according to still one another preferred embodiment of the present invention.

### DETAILED DESCRIPTION OF THE PREFERRED EMBODIMENTS

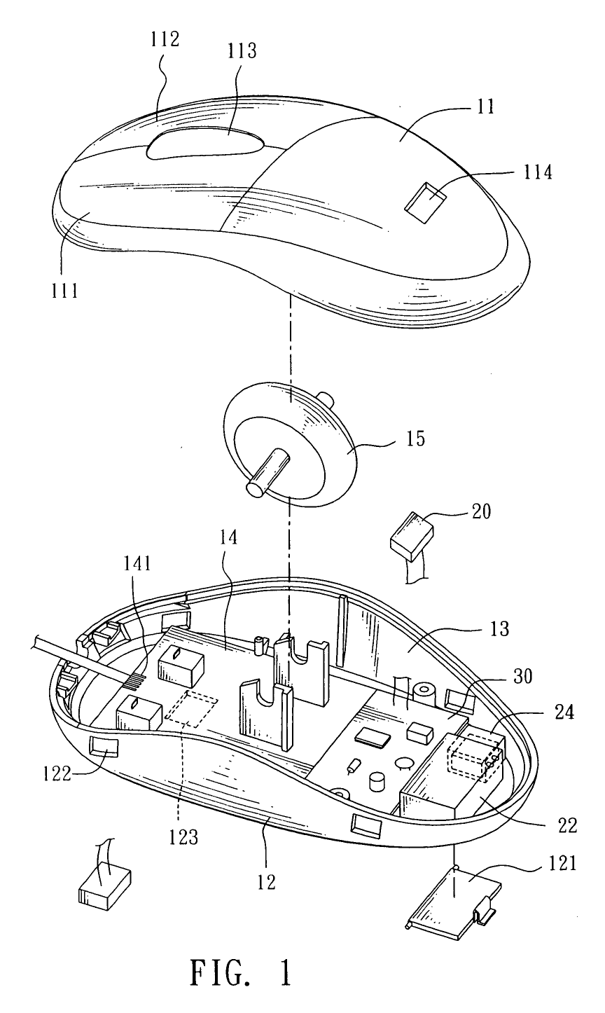

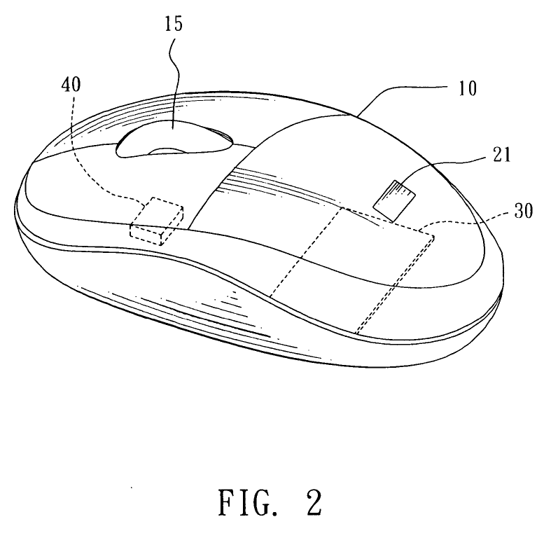

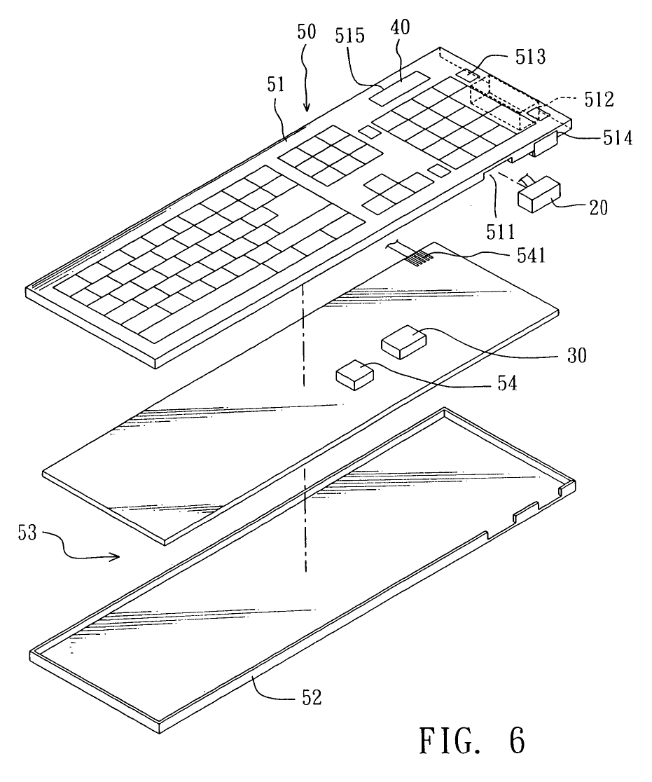

Referring to FIG. 1, which shows a disassembly view of an input device according to one preferred embodiment of the present invention. As shown in FIG. 1, the input device of the present invention comprises: a body 10 (please refer to FIG. 2); a meter 20; and a control circuit 30; wherein the input device of the present invention can be a mouse or a keyboard; for the purpose of explaining, the input device of the present invention shown in FIG. 1-5 is a mouse, preferably; while the input device of the present invention shown in FIG. 6-10 is a keyboard, preferably.

Wherein, the body 10 is formed by a pair of upper cover 11 and lower cover 12 made of hard high polymer material, for example but not limited to ABS or PC by way of molding, and a space 13 formed between the upper cover 11 and lower cover 12 for disposing an electronic circuit 14 with a connecting interface 141 for example but not limited to PS/2, USB or wireless interface, these interfaces are prior arts and used in general computer peripheral products so the description will not describe more detail here. Furthermore, the upper cover 11 comprises at least one left button 111, a right button 112, these button 111, 112 are also prior arts so the description will not describe more detail here too. The upper cover 11 also comprises a groove 113 for providing the scrolling wheel 15 to expose such that the user can operate it conveniently. The upper cover 11 further comprises a first opening 114, while the lower cover 12 comprises a removable bottom cover 121, four second openings 122 and a third opening 123.

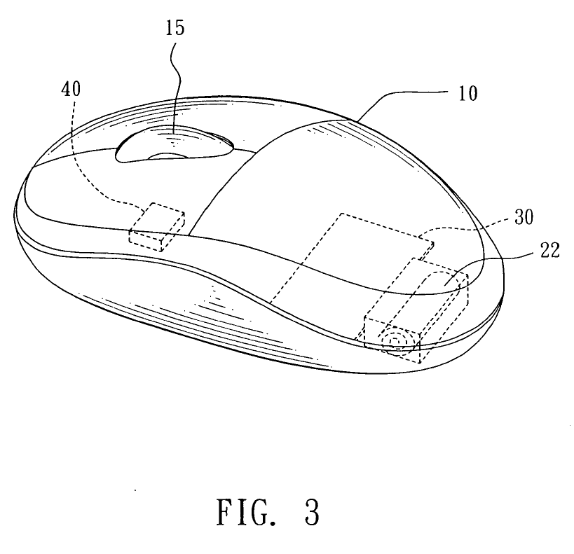

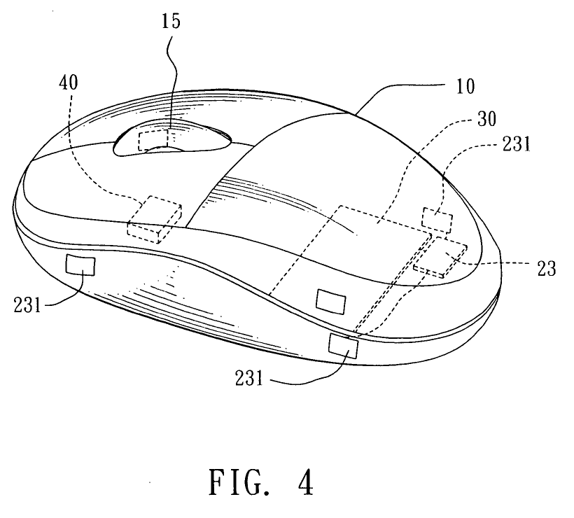

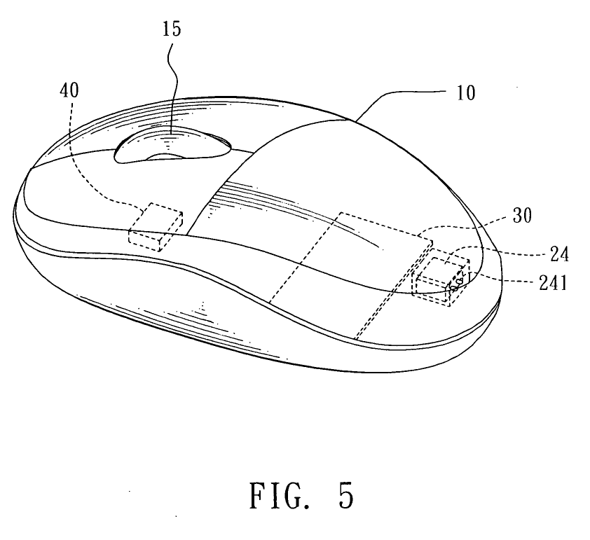

The meter 20 disposed in the body 10 of the present invention can be a temperature meter 21 (please refer to FIG. 2), a blood pressure and heartbeat meter 22 (please refer to FIG. 3), a body fat meter 23 (please refer to FIG. 4) or a skin analyzer 24 (please refer to FIG. 5), such that the input device of the present invention has the ability of measuring the temperature, blood pressure and heartbeat, body fat or skin quality etc. physiology parameters of the user.

Wherein the control circuit 30 is disposed in the space 13 and coupled to the meter 20 and the electronic circuit 14 for receiving the temperature, blood pressure and heartbeat, body fat or skin quality etc. physiology parameters sensed by the meter 20 and generating corresponding actives such as transmitting the physiology parameters sensed by the meter 20 to the monitor program installed on the host computer (figure not shown) through the connecting interface 141 for displaying, monitoring and managing the physiology parameters of the user.

Furthermore, the input device of the present invention further comprises a display apparatus 40 (please refer to FIG. 2) coupled to the control circuit 30 as well as disposed in the space 13 and exposed from the third opening 122; wherein the display apparatus 40 is for example but not limited to a LCD or LED display. When the host computer is shut down or in a place where it doesn't exist any host computer, the display apparatus 40 can display the physiology parameters measured by the meter 20 such that the display apparatus 40 provides another display choice to the user.

Referring to FIG. 2, which shows an assembly view of a mouse with temperature sensing function according to one preferred embodiment of the present invention. As shown in FIG. 2, when the meter 20 of the present invention is a temperature meter 21 for example but not limited to a contact meter or an infrared meter, the input device of the present invention will become a mouse with temperature sensing function; wherein, the temperature meter 21 is disposed in the space 13 and exposed from the first opening 114. The user can take the mouse up and close to the forehead to measure the more precise temperature and the mouse of the present invention will transmit the temperature signal sensed by the temperature meter 21 to the control circuit 30 for processing and then transmits it to the monitor program of the host computer through the connecting interface 141 for displaying or outputs to the display apparatus 40 for displaying.

Referring to FIG. 3, which shows an assembly view of a mouse with blood pressure and heartbeat sensing function according to another preferred embodiment of the present invention. As shown in FIG. 3, when the meter 20 of the present invention is a blood pressure and heartbeat meter 22, the input device of the present invention will become a mouse with blood pressure and heartbeat sensing function; wherein, the blood pressure and heartbeat meter 22 is disposed in the space 13, whereby the blood pressure and heartbeat meter 22 can be separated from the body 10 through the removable bottom cover 121 (please refer to FIG. 1). The user can open the removable bottom cover 121 and take the blood pressure and heartbeat meter 22 out then wears it on a hand to measure the blood pressure and heartbeat signal and the mouse of the present invention will transmit the blood pressure and heartbeat signal sensed by the blood pressure and heartbeat meter 22 to the control circuit 30 for processing and then transmits it to the monitor program of the host computer through the connecting interface 141 for displaying or outputs to the display apparatus 40 for displaying.

Referring to FIG. 4, which shows an assembly view of a mouse with body fat sensing function according to still one another preferred embodiment of the present invention. As shown in FIG. 4, when the meter 20 of the present invention is a body fat meter 23, the input device of the present invention will become a mouse with body fat sensing function; wherein, the body fat meter 23 has four sensing points 231 disposed in the four second openings 122 and exposed from the second openings 122, respectively. The user can put his/her four fingers belonged to two respective hands ( such as the thumb and forefinger of the left and right hand) on the four sensing points 231, respectively, to measure the body fat signal and the mouse of the present invention will transmit the body fat signal sensed by the body fat meter 23 to the control circuit 30 for processing and then transmits it to the monitor program of the host computer through the connecting interface 141 for displaying or outputs to the display apparatus 40 for displaying.

Referring to FIG. 5, which shows an assembly view of a mouse with skin sensing function according to still one another preferred embodiment of the present invention. As shown in FIG. 5, when the meter 20 of the present invention is a skin analyzer 24, the input device of the present invention will become a mouse with skin analyzing function; wherein, the skin analyzer 24 comprises two contacts 241 disposed in the space 13 and can be rotated and protruded from the bottom cover 12. The user can turn the bottom cover 12 and let the contacts 241 be protruded from the body 10 and contact with the skin so as to analyze the skin information and the mouse of the present invention will transmit the skin information analyzed by the skin analyzer 24 to the control circuit 30 for processing and then transmits it to the monitor program of the host computer through the connecting interface 141 for displaying or outputs to the display apparatus 40 for displaying.

Furthermore, the input device of the present invention can be a keyboard with a connecting interface for example but not limited to a PS/2, USB or wireless interface, too. Referring to FIG. 6, which shows a disassembly view of a keyboard according to one preferred embodiment of the present invention. As shown in FIG. 6, the keyboard of the present invention comprises: a body 50; a meter 20; and a control circuit 30; wherein the meter 20 and the control circuit 30 are same as the meter 20 and the control circuit 30 of FIG. 1-5, so the description related to the meter 20 and the control circuit 30 please refer to the aforesaid description.

Wherein, the body 50 is formed by a pair of upper cover 51 and lower cover 52 made of hard high polymer material, for example but not limited to ABS or PC by way of molding, and a space 53 formed between the upper cover 51 and lower cover 52 for disposing an electronic circuit 54 with a connecting interface 541 for example but not limited to PS/2, USB or wireless interface. Furthermore, the side of the upper cover 51 comprises a first opening 511, a first removable side cover 512, four second openings 513, a second removable side cover 514 and a third opening 515. Additionally, the keyboard of the present invention further comprises a display apparatus 40 coupled to the control circuit 30 as well as disposed in the space 53 and exposed from the third opening 515; wherein, the display apparatus 40 is a LCD or LED display.

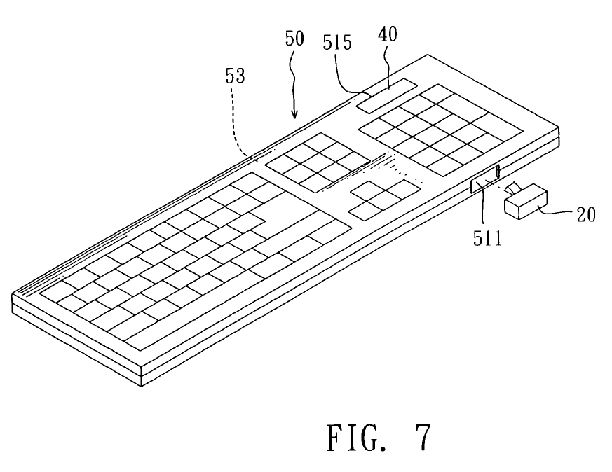

Referring to FIG. 7, which shows an assembly view of a keyboard with temperature sensing function according to another preferred embodiment of the present invention. As shown in FIG. 7, when the meter 20 of the present invention is a temperature meter 21 for example but not limited to a contact meter or an infrared meter, the input device of the present invention will become a keyboard with temperature sensing function; wherein, the temperature meter 21 is disposed in the space 53 and exposed from the first opening 511. The user can take the temperature meter 21 out from the first opening 511 and close to the forehead to measure the more precise temperature and the keyboard of the present invention will transmit the temperature signal sensed by the temperature meter 21 to the control circuit 30 for processing and then transmits it to the monitor program of the host computer through the connecting interface 141 for displaying or outputs to the display apparatus 40 for displaying.

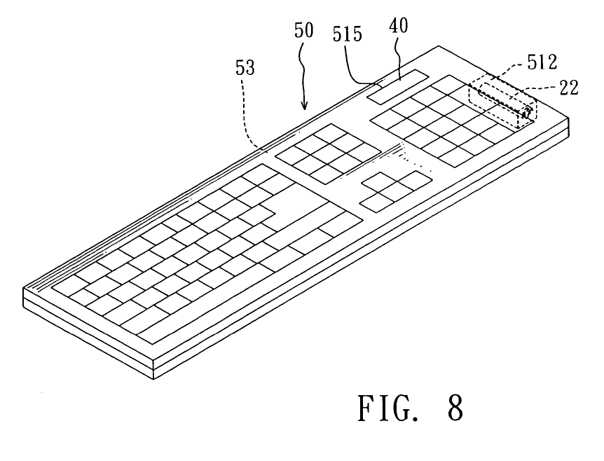

Referring to FIG. 8, which shows an assembly view of a keyboard with blood pressure and heartbeat sensing function according to still one another preferred embodiment of the present invention. As shown in FIG. 8, when the meter 20 of the present invention is a blood pressure and heartbeat meter 22, the input device of the present invention will become a keyboard with blood pressure and heartbeat sensing function; wherein, the blood pressure and heartbeat meter 22 is disposed in the space 53, whereby the blood pressure and heartbeat meter 22 can be separated from the body 50 through the first removable side cover 512. The user can open the first removable side cover 512 and take the blood pressure and heartbeat meter 22 out then wears it on a hand to measure the blood pressure and heartbeat signal and the keyboard of the present invention will transmit the blood pressure and heartbeat signal sensed by the blood pressure and heartbeat meter 22 to the control circuit 30 for processing and then transmits it to the monitor program of the host computer through the connecting interface 141 for displaying or outputs to the display apparatus 40 for displaying.

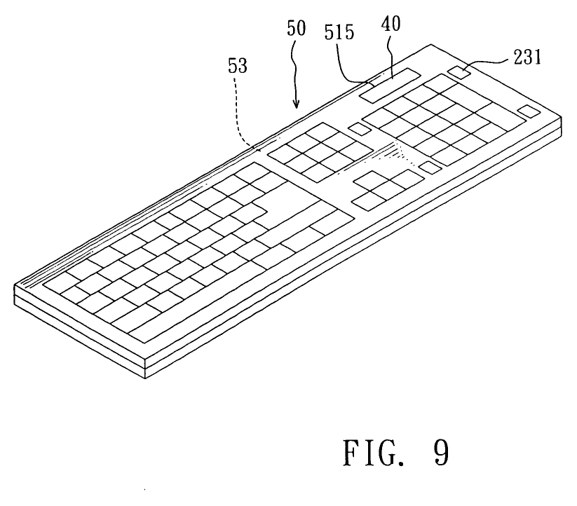

Referring to FIG. 9, which shows an assembly view of a keyboard with body fat sensing function according to still one another preferred embodiment of the present invention. As shown in FIG. 9, when the meter 20 of the present invention is a body fat meter 23, the input device of the present invention will become a keyboard with body fat sensing function; wherein, the body fat meter 23 has four sensing points 231 disposed in the four second openings 513 and exposed from the second openings 513, respectively; wherein, the four sensing points 231 are disposed for example but not limited to around the square digit keypad. The user can put his/her four fingers belonged to two respective hands (such as the thumb and forefinger of the left and right hand) on the four sensing points 231, respectively, to measure the body fat signal and the keyboard of the present invention will transmit the body fat signal sensed by the body fat meter 23 to the control circuit 30 for processing and then transmits it to the monitor program of the host computer through the connecting interface 141 for displaying or outputs to the display apparatus 40 for displaying.

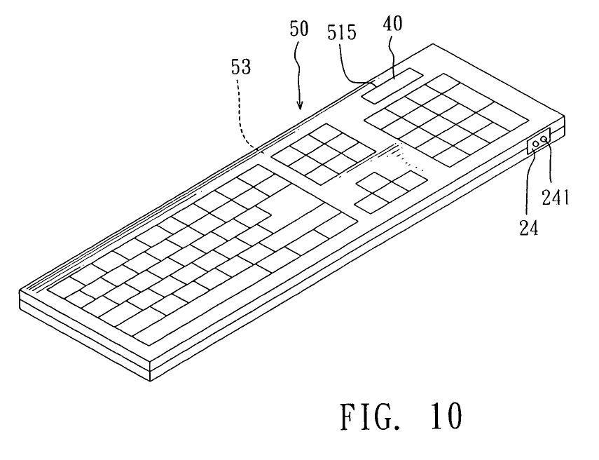

Referring to FIG. 10, which shows an assembly view of a keyboard with skin analyzing function according to still one another preferred embodiment of the present invention. As shown in FIG. 10, when the meter 20 of the present invention is a skin analyzer 24, the input device of the present invention will become a keyboard with skin analyzing function; wherein, the skin analyzer 24 comprises two contacts 241 disposed in the space 53 and can be rotated and protruded from the second removable side cover 514. The user can turn the second removably side cover 514 and let the contacts 241 be protruded from the body 50 and contact with the skin so as to measure the skin information and the keyboard of the present invention will transmit the skin information analyzed by the skin analyzer 24 to the control circuit 30 for processing and then transmits it to the monitor program of the host computer through the connecting interface 141 for displaying or outputs to the display apparatus 40 for displaying.

While the invention has been described with reference to preferred embodiment thereof, it is to be understood that modifications or variations may be easily made without departing from the spirit of this invention, which is defined by the appended claims.

What is claimed is:

1. An input device comprising:
a body, having an upper cover as well as a lower cover and a space formed between them for disposing an electronic circuit with a connecting interface;
a meter, disposing in said body; and
a control circuit, disposing in said space and coupling to said meter and said electronic circuit;
whereby the human physiology parameter detected by said meter can be transmitted to a monitor program installed on a computer through said connecting interface.

2. The input device as claimed in claim 1, wherein said input device is a mouse, preferably.

3. The input device as claimed in claim 2, wherein it further comprises a first opening disposed on a top surface of said upper cover, while a removable bottom cover four second openings and a third opening disposed on said lower cover, respectively.

4. The input device as claimed in claim 3, wherein said meter is a temperature meter exposed from said first opening.

5. The input device as claimed in claim 4, wherein said meter is a contact meter or an infrared meter.

6. The input device as claimed in claim 3, wherein said meter is a blood pressure and heartbeat meter disposed in said space, whereby said blood pressure and heartbeat meter can be separated from said body through said removable bottom cover.

7. The input device as claimed in claim 3, wherein said meter is a body fat meter with four sensing points, wherein, said four sensing points are disposed in said four second openings and exposed from said second openings, respectively.

8. The input device as claimed in claim 3, wherein said meter is a skin analyzer disposed in said space, and said skin analyzer can be rotated and protruded from said bottom cover.

9. The input device as claimed in claim 3, wherein it further comprises a display apparatus coupled to said control circuit as well as disposed in said space and exposed from said third opening.

10. The input device as claimed in claim 9, wherein said display apparatus is a LCD or LED display.

11. The input device as claimed in claim 1, wherein said connecting interface is a PS/2, USB or wireless connecting interface.

12. The input device as claimed in claim 1, wherein said input device is a keyboard, preferably.

13. The input device as claimed in claim 12, wherein it further comprises a first opening, a first removable side cover, four second openings, a second removable side cover and a third opening disposed on said upper cover, respectively.

14. The input device as claimed in claim 13, wherein said meter is a temperature meter exposed from said first opening, whereby said temperature meter can be separated from said body through said first opening.

15. The input device as claimed in claim 14, wherein said meter is a contact meter or an infrared meter.

16. The input device as claimed in claim 13, wherein said meter is a blood pressure and heartbeat meter disposed in said space, whereby said blood pressure and heartbeat meter can be separated from said body through said first removable side cover.

17. The input device as claimed in claim 13, wherein said meter is a body fat meter with four sensing points, wherein, said four sensing points are disposed in said four second openings and exposed from said second openings, respectively.

18. The input device as claimed in claim 13, wherein said meter is a skin analyzer disposed in said space, whereby, said skin analyzer can be separated from said body through said second removable side cover.

19. The input device as claimed in claim 13, wherein it further comprises a display apparatus coupled to said control circuit as well as disposed in said space and exposed from said third opening.

20. The input device as claimed in claim 19, wherein said display apparatus is a LCD or LED display.
